(19) United States
(12) Patent Application Publication
(10) Pub. No.: US 2010/0324336 A1
(43) Pub. Date: Dec. 23, 2010

(54) PROCESS FOR THE PRODUCTION OF AROMATIC AMINES

(75) Inventors: Knut Sommer, Krefeld (DE); Peter Lehner, Mulheim/Ruhr (DE); Andre Lago, Hamburg (DE)

Correspondence Address: BAYER MATERIAL SCIENCE LLC, 100 BAYER ROAD, PITTSBURGH, PA, 15205, US

(73) Assignee: Bayer MaterialScience AG, Leverkusen (DE)

(21) Appl. No.: 12/814,536

(22) Filed: Jun. 14, 2010

(30) Foreign Application Priority Data
Jun. 18, 2009 (DE) 10 2009 025 374.2

(51) Int. Cl. C07C 209/36; C07C 209/84

(52) U.S. Cl. 564/420

(57) ABSTRACT

Aromatic amines produced by hydrogenation of the corresponding nitroaromatic compounds are purified in a specified manner. In the purification procedure, the particular amine is initially mixed with an aqueous solution of a base. The organic and aqueous phases are then separated by adding excess water.

The present invention relates to a process for the production of aromatic amines by hydrogenation of the corresponding aromatic compounds containing nitro groups (“nitroaromatic compounds”) and subsequent purification. Aromatic amines are understood here as meaning compounds carrying at least one amino group on an aromatic ring, it being possible, if desired, for the ring to be substituted or to be fused with other aromatic rings.

These aromatic amines are preferably purified by a procedure in which the particular amine is initially mixed with the smallest possible amount of an aqueous solution of a base that is as concentrated as possible, so that preferably there is not yet a clearly visible phase separation at this stage. Only then are the organic and aqueous phases deliberately separated by adding excess water. The position of the organic phase (top or bottom) is preferably adjusted specifically by choosing appropriate physical boundary conditions.

In the process of the present invention, the crude aromatic amine(s) can be prepared by processes known in the art. One such known process for the production of toluenediamine is disclosed in EP-A-1935871. In this disclosed process, the hydrogenation of the nitroaromatic compound(s) is carried out at temperatures of 100 to 200° C., preferably of 120 to 180° C., more preferably of 125 to 170° C. and most preferably of 130 to 160° C., in the presence of catalysts, at absolute pressures of 5 to 100 bar, preferably of 8 to 50 bar and most preferably of 10 to 35 bar.

In principle, the nitroaromatic compound(s) used can be any of those conventionally used in industry. It is preferable to use aromatic mono-nitro and/or di-nitro compounds and particularly preferable to use nitrobenzene, nitrotoluene and dinitrotoluene.

One example of a reaction apparatus suitable for conducting the hydrogenation reaction is the slurry phase reactor described in WO-A-96/11052. Other suitable reactors are described in EP-A-0236935 and U.S. Pat. No. 6,350,911. It is, of course, also possible to use several identical reaction apparatuses or combinations of different suitable reaction apparatuses.

The catalyst(s) used to conduct the hydrogenation of nitroaromatic compounds in accordance with the present invention can be any of the hydrogenation catalysts known to be useful for the catalytic hydrogenation of nitroaromatic compounds. Particularly suitable catalysts are the metals of subgroup 8 of the Periodic Table of the Elements, or mixtures thereof, which may optionally be applied to a support such as carbon or oxides of magnesium, aluminium and/or silicon. It is preferable to use Raney iron, cobalt and/or nickel, especially nickel-containing catalysts such as Raney nickel catalysts, and palladium or platinum-containing catalysts on supports. The preparation and use of these preferred catalysts for the hydrogenation of nitroaromatic compounds, e.g., nitrobenzene, nitrotoluenes, dinitrotoluenes, chlorinated nitroaromatics, etc., are known and have been described in, for example, EP-A-0223035, EP-B-1066111, and EP-A-1512459. The processes described in these EP disclosures are particularly significant for the hydrogenation of dinitrotoluene.

Aromatic amines are important intermediates that have to be available at an economic price and in large quantities. Therefore, plants of very large capacities have to be built for the manufacture of amines such as aniline. Aniline, for example, is an important intermediate in the manufacture of methylenediphenyl diisocyanate (MDI) and is normally manufactured on a large scale by the catalytic hydrogenation of nitrobenzene, as described, e.g., in DE-A-2201528, DE-A-3414714, U.S. Pat. No. 3,136,818, EP-B-0696573, EP-B-0696574 and EP-A-1882681. In principle, in the case of aniline, the aniline can originate from any of the nitrobenzene hydrogenation processes conventionally used in industry. Preferably, the hydrogenation of nitrobenzene is carried out in the gas phase on fixed, heterogeneous supported catalysts (e.g., Pd on aluminium oxide or carbon supports) in fixed bed reactors, at an absolute pressure of 2-50 bar and a temperature ranging from 250 to 500° C., under adiabatic conditions, in a gas recycling operation (i.e., with recycling of the hydrogen that has not reacted during the hydrogenation (cf. EP-A-0696573 and EP-A-0696574)).

In the preparation of one or more aromatic amine(s), water and organic by-products are formed in addition to the target products. These organic by-products have to be separated off before the aromatic amine(s) is/are used. The separation of by-products whose boiling points are very similar to that of the amine to be prepared is particularly problematic because the distillation costs are substantial. In the case of the preparation of aniline (b.p.=184° C.), the separation of phenol (b.p.=182° C.), in particular, makes great demands on the distillation technology, and this is reflected in the use of long distillation columns having a large number of separation stages and high reflux ratios, with correspondingly high investment and energy costs. Compounds with phenolic hydroxyl groups (i.e., compounds carrying at least one hydroxyl group on an aromatic ring) can generally be problematic in the working-up of aromatic amines. In the case of aniline, the various aminophenols, in particular, may be mentioned in addition to the phenol mentioned above.

The purification of aromatic amines is therefore not a trivial matter and is of great industrial importance. More recent attempts to purify aromatic amines have been particularly concerned with phenolic hydroxyl groups. In the attempted solution, the compounds with phenolic hydroxyl groups are converted into the corresponding salts by reaction with suitable bases. As non-volatile compounds, these salts can be separated off much more easily.

JP-A-49-035341 describes a process in which the amine to be purified, namely aniline, is brought into contact with solid alkali metal hydroxides in a fixed bed and only then passed on for distillation. In an alternative embodiment, the distillation is carried out in the presence of the solid alkali metal hydroxide in proportions of 0.1-3 wt. %, based on the amount of aniline to be distilled. This simplifies the separation of critical components like the aminophenols. However, the disadvantages of this process are the use of high molar excesses of the solid alkali metal hydroxides in relation to the acidic secondary components to be removed, and the fact that it is impossible to dose the alkaline compounds accurately. This can lead on the one hand (overdosing) to corrosion problems, precipitation and high-viscosity bottom phases in the distillation column, and on the other hand (underdosing) to an incomplete removal of the critical components.

U.S. Published Patent Application 2005 080294 describes a process for the separation of compounds with phenolic hydroxyl groups (referred to as phenolic compounds) from aromatic amines in which, prior to the distillation, a base is added to the amine to be purified in a molar ratio of 1:1 to 4:1, based on the phenolic compounds, optionally in the presence of polyols. U.S. 2005 080294 does not specifically teach what happens to the salts formed in the reaction of the phenolic compounds with the bases. It is merely mentioned, in Example 6, that excess solid KOH is solubilized by the addition of polyethylene glycol (PEG), but U.S. 2005 080294 does not state what consequences are associated with this action. U.S. 2005 080294 gives no details with respect to the salts of the phenolic compounds themselves.

However, salts, meaning not only excess base but also the salts of the compounds with phenolic hydroxyl groups, are generally only poorly soluble in aromatic amines, so there is a great danger that they will accumulate beyond the solubility limit in the distillation column, in the bottom of the distillation column and/or in the evaporator, and then precipitate. Such solid deposits can so severely interfere with the distillation process that it becomes necessary to stop it, which, in continuous, large-scale production, can cause considerable difficulties and even loss of production. However, U.S. 2005 080294 does not address the problem of the reliability and operating life of the process. U.S. 2005 080294 also fails to teach those skilled in the art that the presence of the salts formed during the reaction between the compounds with phenolic hydroxyl groups and the bases can cause the deposition of solids, fouling and/or a sharp increase in viscosity during the distillation. U.S. 2005 080294 gives no details with respect to the distillation technique, so it does not teach those skilled in the art how they are supposed to solve these problems that occur with a high probability. U.S. 2005 080294 teaches only the optional addition of PEG to solubilize excess solid KOH. However, such an addition of PEG to the distillation is economically disadvantageous because of the high capacities used in the manufacture of aromatic amines (especially aniline).

EP-A-1845079 describes a process for the purification of aniline by adding an aqueous alkali metal hydroxide solution before or during the distillation. The problems due to the deposition of solids, fouling and/or a sharp increase in viscosity during the distillation are prevented by partially discharging the bottom phase of the distillation, washing it with water or dilute alkali metal hydroxide solution and recycling the washed organic phase into the distillation. The disadvantage of this process is that an additional process step is needed to maintain a reliable operation.

EP-A-2028176 describes a process for the purification of aromatic amines in which the crude amine obtained after separation of the process water is treated with aqueous alkali metal hydroxide solution and the resulting process product is distilled. The bottom of the distillation column is partially or completely discharged and part of it is vaporized by means of two evaporators (E¹) and (E²) connected in series or parallel. This is said to achieve a maximum depletion of the valuable amine in the bottom of the distillation column with minimal expenditure on apparatus and energy.

In all of the processes mentioned thus far, the aromatic amine is distilled in the presence of a base. In such a procedure, problems due to the deposition of solids, fouling and/or a sharp increase in viscosity during the distillation have to be prevented by laborious and/or costly means.

As an alternative to the removal of compounds with phenolic hydroxyl groups from aniline during the distillation, JP-A-08-295654 describes an extraction with dilute aqueous alkali metal hydroxide solution. In one preferred embodiment, described only in Examples 1 to 5, this is done by a procedure in which the aqueous phase obtained after mixing the phenol-containing aniline with the alkali metal hydroxide solution contains the alkali metal hydroxide in a concentration of 0.1 to 0.7 wt. %, and the organic phase obtained after phase separation is distilled. Most of the phenol is transferred to the aqueous phase as alkali metal phenolate in the extraction step and is thereby separated in the phase separation from the aniline to be purified. In Examples 1 to 5 of JP-A-08-295654, the product of nitrobenzene hydrogenation is treated directly (i.e., without prior separation of the process water) with aqueous sodium hydroxide solution at a molar ratio of NaOH to phenol of at least 69:1 (Example 2). Restriction to the low concentration of ≦0.7 wt. % is said to improve the phase separation. The disadvantages of this process are the high consumption of NaOH and the production of very large amounts of effluent containing alkali metal phenolate, as a result of the low concentration of the alkali metal hydroxide solutions.

EP-A-1845080 describes a process for the purification of aniline by extraction with aqueous alkali metal hydroxide solution having a concentration of >0.7 wt. %. The concentration and temperature are adjusted so that the aqueous phase always forms the bottom phase in the subsequent phase separation. This assures a stable operation because no problems arise due to phase reversal. Also, this process is carried out only with relatively low concentrations of alkali metal hydroxide solution (between 0.8 and 2.5 wt. %, based on the weight of alkali metal hydroxide solution, in the Examples) and a high molar excess of alkali metal hydroxide (at least 12.52:1 in the Examples). The weight ratio of organic to aqueous fraction in the extraction is only 5.0 or less in the Examples.

JP-A-2007217405 describes a process in which the phenol-containing aniline is brought into contact at least twice with aqueous alkali metal hydroxide solution in such a way that the concentration of alkali metal hydroxide in the aqueous phase is between 0.1 and 0.7 wt. %. This is achieved by adding first water and then a relatively concentrated (25%) aqueous NaOH solution to the crude aniline obtained after phase separation (cf. Examples). The molar excesses of alkali metal hydroxide, based on phenol, used in the Examples, always given as the total over both extraction steps, are at least 10.6. This can be taken from Example 3, where 174 g of a crude aniline with a phenol content of 522 ppm (corresponding to 0.966 mmol of phenol) are purified and 2×0.82 g of 25% NaOH solution (corresponding to 10.25 mmol of NaOH in total) are added. Thus, in this process too, a relatively large molar excess of alkali metal hydroxide is required, with all the disadvantages already mentioned in the discussion of JP-A-08-295654.

In all of the above-described processes for the purification of aromatic amines using an extractive base treatment, the only bases that are economically viable for large-scale production are those which are available in large quantities at an economic price. In practice, therefore, aqueous solutions of alkali metal hydroxides, especially sodium hydroxide, are nearly always chosen. According to the state of the art, the base is added in the form of a relatively highly dilute aqueous solution or the amine to be purified is treated with water before the base is added, or the process water is not separated off, so the concentration of base in the aqueous fraction of the resulting process product is low and the weight ratio of organic to aqueous fraction is relatively small. For example, the weight ratio of organic fraction to aqueous fraction in the first extraction step of Example 3 in JP-A-2007217405 is only 2.6 (174 g of crude aniline to 67 g of water and 0.82 g of NaOH solution). This calculation has not taken into account the fact that the crude aniline used can contain several percent of water, so the actual value is probably even smaller. Because of the poor miscibility of the aqueous base-containing fraction with the crude aromatic amine, the reaction of acidic impurities with particles of base only takes place at the phase interface, so it is necessary to use very large molar excesses of base.

The ideal base from the point of view of reactivity would be an organic base that has a good miscibility with the aromatic amine. This could be used in the stoichiometric amount and the salt formed could then be separated off easily. However, since this method is not economically viable in large-scale production, other solutions to the described problem must be sought.

### SUMMARY OF THE INVENTION

The object of the present invention was to provide a process for the production and purification of aromatic amines which allows the separation of compounds with phenolic hydroxyl groups by a treatment with base(s) in such a way that
it can be carried out with minimal molar excesses of the base used, relative to the acidic compounds to be removed,
a stable operation is assured without the risk of a phase reversal during the separation of organic and aqueous phases, and
problems such as the deposition of solids, fouling and/or a sharp increase in viscosity during the distillation are avoided.

This and other objects which will be apparent to those skilled in the art are accomplished by (1) mixing the crude aromatic amine with at least one aqueous solution of a base in a specified amount and concentration; (2) mixing the mixture from (1) with water in an amount such that a specified weight ratio is achieved; and (3) separating the two-phases of the mixture from (2).

### DETAILED DESCRIPTION OF THE PRESENT INVENTION

The present invention relates to a process for the preparation of one or more aromatic amines in which:
a) the aromatic amine(s) is/are prepared by hydrogenation of the corresponding nitroaromatic compound(s) in the presence of a catalyst, and the water formed in the hydrogenation (process water) is separated off by phase separation to give the crude aromatic amine(s), and
b) the crude aromatic amine(s) obtained from step a) is/are then purified by a procedure in which
   b) (i) the crude aromatic amine(s) obtained from step a) is/are mixed with at least one aqueous solution of a base, the amount of base used and the base concentration of the aqueous base solution must be such that the molar ratio of the sum of all the base(s) added to the aromatic amine(s) in process step b) to the phenolic hydroxyl groups contained in the aromatic amine(s) obtained from step a) is not greater than 10, and is preferably between 1 and 10, and such that the weight ratio of organic constituents to water is greater than 10,
   b) (ii) the mixture obtained from step b) (i) is mixed with water in an amount such that the weight ratio of the mixture obtained from step b) (i) to the water added in step b) (ii) is between 0.05 and 20, preferably between 0.1 and 10 and most preferably between 0.2 and 5, and
   b) (iii) the two-phase mixture obtained from step b) (ii) is separated into an organic phase containing the aromatic amine(s) and an aqueous phase.

Preferably, the temperature and pressure in the phase separation in step b) (iii) are chosen so that the organic phase adopts a specific predetermined position (either top or bottom) in the phase separation.

The weight ratio of organic constituents to water required in step b) (i), namely greater than 10, preferably greater than 50, particularly preferably greater than 100 and very particularly preferably greater than 150, means that, overall, it is possible to work with smaller molar excesses of base than has been conventional in the art of extractive purification processes. Conventionally, the weight ratio will not exceed a value of 10,000.

In the process of the process of the present invention, the proportion of aqueous phase (i.e., the phase that is only poorly miscible with the aromatic amine) is greatly reduced compared with the state of the art because the reaction between the acidic impurities and the particles of base is considerably facilitated. Preferably, at this stage of the process, there is no clearly visible phase separation when stirring is stopped. A significant amount of water is only added, in step b) (ii), after completion of the actual acid-base reaction. Water, salts dissolved therein and excess base are then separated off in the subsequent phase separation in step b) (iii).

The crude aromatic amines obtained from step a) and used in step b) (i) can originate from any of the known industrial processes for the manufacture of aromatic amines. Particularly suitable aromatic amines are those obtained by catalytic reduction of the corresponding nitro compounds. Such processes are explained above by way of example for toluenediamine and aniline. The process water obtained in these processes is separated from the organic reaction product by phase separation techniques known to those skilled in the art. The crude amine obtained in this way in step a) preferably still has a residual water content corresponding to the solubility of water in the crude amine under the given physical boundary conditions (especially temperature and pressure). The residual water content may be between 0.01 and 20 wt. %, based on the weight of crude amine (tantamount to the aromatic amine obtained in step a)).

Whether the organic phase forms the top or the bottom phase in the phase separation in step b) (iii) is irrelevant to the functioning of the process. Both situations can be advantageous, depending on the equipment. For example, if it is desired, under the given prerequisites of a production plant, that the organic phase be the bottom phase in the phase separation, the physical boundary conditions, especially the temperature, are adjusted accordingly. The only important point is that a phase reversal does not occur unexpectedly during the process. This is easily avoidable with the process according to the invention.

Depending on the level to which the content of compounds with phenolic hydroxyl groups is to be reduced, the organic phase obtained from step b) (iii), containing the aromatic amine(s), can be subjected to a further base treatment, i.e., process step b) is extended to include further purification stages.

Where further purification stages are to be included, the additional steps may include:
b) (iv) the organic phase obtained from step b) (iii) (containing the aromatic amine(s)) is mixed with at least one aqueous solution of a base in an amount and at a concentration such that the molar ratio of the sum of all the bases added to the aromatic amine(s) in process step b) to the phenolic hydroxyl groups contained in the aromatic amine(s) obtained from step a) is not greater than 10, preferably between 1 and 10, and that the weight ratio of organic constituents to water is greater than 10,
b) (v) the mixture obtained from step b) (iv) is mixed with water in an amount such that the weight ratio of the mixture obtained from step b) (iv) to the water added in step b) (v) between 0.05 and 20, preferably between 0.1 and 10 and most preferably between 0.2 and 5, and
b) (vi) the two-phase mixture obtained from step b) (v) is then separated into an organic phase containing the aromatic amine(s) and an aqueous phase.

Preferably, the temperature and pressure in the phase separation in step b) (vi) are chosen so that the organic phase adopts a specific predetermined position (either top or bottom) in the phase separation.

In principle, further base treatments can then be carried out, although this is not generally necessary.

If required, the organic phase obtained after the phase separation in step b) (iii) or b) (vi), containing the aromatic amine(s), can be washed with water in a one-stage or multistage process in order to remove the last residues of excess base and salts of the compounds with phenolic hydroxyl groups.

The water used in each of the described process steps b) (ii) and b) (v) can originate from any desired source. Thus de-mineralized water, plant water and process water are equally suitable. For economic reasons, it is preferable to use plant water and most preferable to use process water. Process water is understood as meaning the water formed in the preparation of the aromatic amines in step a). It can either be used in the form obtained after the phase separation, or after purification by distillation or other means.

The base used in the process of the present invention is preferably an alkali metal hydroxide, most preferably sodium hydroxide or potassium hydroxide. It is also conceivable, in principle, to use other water-soluble basic compounds. The concentration and amount of the aqueous base solution added are chosen so that the required parameters in step b) (i) or b) (iv), with respect to the weight ratio of organic to aqueous fraction and with respect to the molar excess of base, are maintained. For this purpose, it is necessary to determine the concentration of the compounds with phenolic hydroxyl groups in the amine to be purified. This is preferably done by customary analytical methods, particularly preferably gas chromatography. The base solution used should preferably be as concentrated as possible. The maximum usable concentration is limited only by the solubility limit of the base in water under the given conditions. It is preferable to use a solution of sodium hydroxide in water, the proportion of sodium hydroxide by weight being preferably at least 10%, more preferably at least 20% and even more preferably at least 30%, based on the total weight of sodium hydroxide solution. It is most preferable to use commercially available 32% sodium hydroxide solution.

Depending on the concentration of the aqueous base solution, the temperature in step b) (i) and/or b) (iv) is preferably between 20° C. and 160° C., more preferably between 30° C. and 100° C. and most preferably between 50° C. and 95° C. The temperature is preferably in the same range during steps b) (ii) and b) (iii) or b) (v) and b) (vi) and, if appropriate, during the final washing step.

In addition to achieving a specific position of the organic phase in the phase separation, the choice of an appropriate combination of the concentration of the aqueous base solution and the temperature during the extraction depends on the process engineering and economic criteria relevant to the particular process. Thus, it may be useful to minimize the temperature in order to limit the water solubility of the aromatic amine. It may, however, be advantageous in process engineering terms to condense the crude amine at elevated temperature after the reaction and then to extract it as well at the same temperature. It may also be useful to enhance the efficacy of the thorough mixing in step b) (i) or b) (iv) by raising the temperature.

Any of the methods and apparatuses known to those skilled in the art for the mixing of liquid phases can be used to mix the aqueous base into the aromatic amine (steps b) (i) and b) (iv)). Examples of suitable apparatus include: static mixers, nozzles, mixing pumps and stirrers. Any of the extraction methods and apparatuses known to those skilled in the art can be used for the extractive washes (steps b) (ii) and b) (iii) or b) (v) and b) (vi)) and for any other necessary washing stages. Examples of suitable extraction apparatus include mixer-settlers and extraction columns. The extractive base treatment can be carried out in co-current or counter-current. In one preferred embodiment of the present invention, a two-stage mixer-settler is used in counter-current for the extractive base treatment. To shorten the necessary separation and residence times, the separators can be provided with coalescence aids such as knitted fabrics, plates or packing.

All the above-described prior art processes for the purification of aromatic amines in which the base is present in the crude amine during a distillation suffer from problems such as the deposition of solids, fouling and/or a sharp increase in viscosity during the distillation. If required, the aromatic amines can also be further purified by distillation in the process according to the invention, in addition to the extractive base treatment. This distillation can be carried out either before or after the base treatment (or, if appropriate, after the final wash). However, deposition of solids, fouling and/or a sharp increase in viscosity do not present difficulties in the process of the present invention because excess base and the salts of the compounds with phenolic hydroxyl groups (i.e., the cause of these problems) are not present during the distillation in the process according to the invention, either because the distillation is carried out before the base treatment or because the inorganic constituents are separated off in step b) (iii) or b) (vi) and/or, if appropriate, in the final wash.

The downstream or upstream distillation steps can take the form of any of the variants familiar to those skilled in the art and be operated under a very wide variety of conditions. Thus a distillation may be carried out, for example, in one or more plate columns or packed columns, or else in divided wall columns. Separation of low and high boilers may take place in different columns or in one column with discharge of the aromatic amine as a side stream.

In principle, the process according to the invention can be applied to any aromatic amines. The amine to be purified can originate from any of the processes conventionally used in industry for the manufacture of aromatic amines. The process according to the invention is particularly suitable for the purification of aniline. Using processes known from the state of the art, the purified aniline can then be reacted with formaldehyde, in the presence of an acidic catalyst, to give diamines and polyamines of the diphenylmethane series. Again using processes known from the state of the art, the diamines and polyamines can then be reacted, preferably with phosgene, to give the corresponding diisocyanates and polyisocyanates of the diphenylmethane series.

The successful implementation of the extractive base treatment of an aromatic amine with ratios of organic to aqueous fraction such as those required in the process of the present invention is surprising in view of the prior art because to date it has been assumed that use of such ratios would result in a drop in extraction efficiency and a lengthening of separation times (cf. e.g. EP-A-1845080, [0013], 1. 15-17).

### EXAMPLES

The Examples described below were carried out in a double-walled separating funnel fitted with a KPG stirrer. This apparatus was maintained at a constant temperature by passing water at 30° C. through the cavity between the two glass walls.

The material to be purified was a crude aniline having a water content of 1500 ppm (determined by the Karl Fischer method) and a phenol content of 998 ppm (determined by gas chromatography), obtained from the hydrogenation of nitrobenzene on a Pd/Pb-on-aluminum oxide catalyst (analogously to EP 1882681 A1, Example 3).

Solely for reasons of simplification of the laboratory procedure, the organic phases obtained after each of the phase separations were divided up and only a portion of them was passed on to the next processing step. The amounts of NaOH solution and water to be added were adjusted in view of the reduced amount in each case. In a large-scale application, these organic phases would preferably be passed on in their entirety to the next processing stage.

### Example 1

### According to the Invention, Small Amounts of Water Added after Base Treatment

1000 g of the crude aniline (phenol content 998 ppm) were treated with 5.00 g of 32% NaOH solution (weight ratio of organic constituents to water=154) and the resulting mixture was stirred thoroughly for 3 minutes (step b) (i)). 250 g of water were then added and the resulting mixture was stirred thoroughly for 3 minutes (step b) (ii)). This stirred mixture was left to stand until the phases separated. The organic phase formed the bottom phase and there were no problems at all with temporary phase reversal. After the phase separation (step b) (iii)), the phenol content and sodium content of the organic phase were determined (by gas chromatography and atomic absorption spectroscopy, respectively). 800 g of the organic phase (containing approx. 4.6% of water) were treated in the next step with 4.00 g of 32% NaOH solution and the resulting mixture was stirred thoroughly for 3 minutes (step b) (iv)). 200 g of water were then added and the resulting mixture was stirred thoroughly for 3 minutes (step b) (v)). This stirred mixture was left to stand until the phases separated. The organic phase formed the bottom phase and there were no problems at all with temporary phase reversal. After the phase separation (step b) (vi)), the organic phase was analyzed as described above. 600 g of the organic phase obtained after the second phase separation (step b) (vi)) were finally stirred thoroughly for 3 minutes with an additional 160 g of water (wash) and the organic phase obtained after the phase separation was analyzed as described above.

### Example 2

### According to the Invention, Large Amounts of Water Added after Base Treatment

The basic procedure was as described in Example 1 with the exception that 1000 g (instead of 250 g) of water were added in step b) (ii) and 800 g (instead of 200 g) of water were added in step b) (v). 640 g (instead of 160 g) of water were used in the final wash. Once again, no problems at all were observed with temporary phase reversal. The organic phase was always the bottom phase in the phase separations. The organic phases obtained after the phase separation were analyzed in each case as described in Example 1.

The Table below contains a comparative overview of the results of Examples 1 and 2:

TABLE 1
Comparison of the results of the Examples

| | | First base treatment, addition of water | | | | Second base treatment, addition of | | | | |
|---|---|---|---|---|---|---|---|---|---|---|
| | | and phase separation | | | | water and phase separation | | | | |
| | | Steps (i)-(iii) | | | | Steps (iv)-(vi) | | | | Wash and |
| | | Weight | Weight | | | Weight | Weight | | | phase |
| | | ratio or | ratio of | Phenol | Sodium | ratio of | ratio of | Phenol | Sodium | separation |
| | Molar Ratio | organic to | mixture | content of | content of | organic | mixture | content of | content of | Sodium |
| | of total base | aqueous | from step | organic | organic | aqueous | from step | organic | organic | content |
| | used to | fraction in | (i) to | phase | phase | fraction in | (iv) to | phase | phase | after wash |
| | phenol in | mixture | amount of | from step | from step | mixture | amount of | from step | from step | and phase |
| Ex. | crude | from step | water | (iii) | (iii) | from step | water | (vi) | (vi) | separation |
| No. | aniline^{[a]} | (i)^{[b]} | added | [ppm] | [ppm] | (iv)^{[b]} | added | [ppm] | [ppm] | [ppm] |
| 1 | 7.53 | 154 | 4.02 | 150 | 52 | 18.7 | 4.02 | 37 | 95 | <1 |
| 2 | 7.53 | 154 | 1.01 | 178 | <1 | 18.7 | 1.01 | 29 | <1 | <1 |

Legend:
^{[a]}Calculated for complete further processing of the organic phase obtained after a phase separation in the next step.
^{[b]}Taking into account the water content already present in the organic phase before the addition of base.

The Examples show that the very high initial phenol content of nearly 1000 ppm can be reduced by approximately 97% with only small molar excesses of base. The final wash can be omitted when the amount of water with which the mixture from step b) (i) or the mixture from step b) (iv) is treated is sufficiently large. (Example 2: the sodium content of the organic phase is already <1 ppm even without the final wash).

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. Process for the production of an aromatic amine comprising
a) removing water formed during hydrogenation of a nitroaromatic compound by phase separation to obtain a crude aromatic amine, and
b) purifying the crude aromatic amine by a procedure comprising:
b) (i) mixing the crude aromatic amine with at least one aqueous solution of at least one base in an amount and in a concentration such that
(A) a molar ratio of total amount of any base added to the aromatic amine in purifying the crude aromatic amine to amount of phenolic hydroxyl groups contained in the crude aromatic amine from a) not greater than 10 is achieved, and
(B) a weight ratio of organic constituents to water greater than 10 is achieved,
b) (ii) mixing the mixture obtained from b) (i) with water in an amount such that a weight ratio of the mixture obtained from step b) (i) to the water added in step b) (ii) between 0.05 and 20 is achieved, and
b) (iii) separating the mixture obtained from b) (ii) into an organic phase containing the aromatic amine and an aqueous phase.

2. The process of claim 1 in which purifying step b) further comprises the steps:
b) (iv) mixing the organic phase obtained from step b) (iii), containing the aromatic amine with at least one aqueous solution of a base in an amount and in a concentration such that
(A) a molar ratio of total amount of any base added to the aromatic amine in purifying the crude aromatic amine to amount of phenolic hydroxyl groups contained in the crude aromatic amine from a) not greater than 10 is achieved, and
(B) a weight ratio of organic constituents to water greater than 10 is achieved,
b) (v) mixing the mixture obtained from step b) (iv) with water in an amount such that a weight ratio of the mixture obtained from step b) (iv) to the water added in step b) (v) between 0.05 and 20 is achieved, and
b) (vi) separating the mixture obtained from step b) (v) into an organic phase containing the aromatic amine and an aqueous phase.

3. The process of claim 2 in which the organic phase obtained from step b) (iii) or step b) (vi) containing the aromatic amine is washed with water in a one-stage or multistage process.

4. The process of claim 1 in which the organic phase obtained from step b) (iii) containing the aromatic amine is washed with water in a one-stage or multistage process.

5. The process of claim 2 in which steps b) (i)-(iii) and steps b) (iv)-(vi) are carried out at temperatures between 20° C. and 160° C.

6. The process of claim 1 in which steps b) (i)-(iii) are carried out at temperatures between 20° C. and 160° C.

7. The process of claim 1 in which the aromatic amine is aniline.

8. The process of claim 2 in which the aromatic amine is aniline.

9. The process of claim 2 in which the water added in step b) (ii) or step b) (v) is recycled process water obtained from the reaction in step a).

10. The process of claim 1 in which the water added in step b) (ii) is recycled process water obtained from the reaction in step a).

11. The process of claim 1 in which the base used is an alkali metal hydroxide.

12. The process of claim 2 in which the base used is an alkali metal hydroxide.

13. The process of claim 1 in which the crude aromatic amine obtained from step a) has a water content of 0.01 to 20 wt. %, based on the weight of the crude aromatic amine obtained from step a).

14. The process of claim 2 in which the crude aromatic amine obtained from step a) has a water content of 0.01 to 20 wt. %, based on the weight of the crude aromatic amine obtained from step a).

15. The process of claim 2 in which the organic phase obtained after the phase separation in step b) (iii) and/or step b) (vi), or any other phase separation, containing the aromatic amine, is purified further by distillation.

16. The process of claim 1 in which the organic phase obtained after the phase separation in step b) (iii) or any other phase separation, containing the aromatic amine, is purified further by distillation.

17. The process of claim 13 in which the crude aromatic amine obtained from step a) is purified by distillation before step b).

18. The process of claim 14 in which the crude aromatic amine obtained from step a) is purified by distillation before step b).
